(19) United States
(12) Patent Application Publication
(10) Pub. No.: US 2026/0102111 A1
(43) Pub. Date: Apr. 16, 2026

(54) PIGMENTATION-INSENSITIVE IMAGING DEVICES, SYSTEMS, AND METHODS

(71) Applicant: Washington University, St. Louis, MO (US)

(72) Inventors: Leonid Shmuylovich, St. Louis, MO (US); Quinlan McGrath, St. Louis, MO (US); Mikhail Berezin, St. Louis, MO (US)

(21) Appl. No.: 19/357,694

(22) Filed: Oct. 14, 2025

Related U.S. Application Data
(60) Provisional application No. 63/706,904, filed on Oct. 14, 2024.

(51) Int. Cl. A61B 5/00

(52) U.S. Cl. CPC A61B 5/445; A61B 5/0077; A61B 2576/02

(57) ABSTRACT

A method of pigment insensitive dermatological inspection of a portion of a body of a subject using an imaging device operable to capture images in a range of wavelengths of light that includes at short-wave infrared (SWIR) wavelengths includes operating the imaging device to capture a first image, a second image, and a third images of the portion of the body of the subject. The first image is obtained at a first passband centered on a first SWIR wavelength within the range of wavelengths, the second image is obtained at a second passband centered on a second SWIR wavelength within the range of wavelengths, and the third image is obtained at a third passband centered on a third SWIR wavelength within the range of wavelengths. The first, second and third images are combined to create a combined image and the combined image is output.

### CROSS REFERENCE TO RELATED APPLICATIONS

This application claims priority to U.S. Provisional Application No. 63/706,904 filed Oct. 14, 2024, which is hereby incorporated by reference in its entirety.

### STATEMENT REGARDING FEDERALLY SPONSORED RESEARCH & DEVELOPMENT

This invention was made with government support under OD031872 awarded by the National Institutes of Health. The government has certain rights in the invention.

### BACKGROUND

The field of this disclosure relates generally to imaging devices. Specific embodiments of this disclosure relate to devices, systems, and methods for pigmentation-insensitive imaging.

Skin disease affects 1 in 4 Americans and is associated with significant healthcare costs annually. Dermatologists primarily rely upon their eyes to diagnose and monitor disease activity through visible inspection or visible photography. But even to the trained eye, visual inspection is fundamentally limited as a subjective and qualitative rather than quantitative measure of disease.

Some disease quantitative approaches have been developed, but even quantification relies on subjective grading by eye. For example, in a common inflammatory disease like eczema, quantitative assessment typically consists of clinicians grading the perceived degree of redness on a scale of 0 to 3, and this combined with other features like amount of skin affected and the degree of skin thickening is used to generate an overall eczema severity index.

The inaccuracies associated with a purely visual approach to skin disease diagnosis and management are well known to dermatologists, and while there is a need to deliver more objective assessment of skin disease, quantitative tools to track skin disease severity have to date not been established.

The subjective nature of visual assessment impacts non-white patients in particular. Non-white patients make up 40% of the population and the visual appearance of skin disease can be quite different in the presence of skin pigmentation. For example, in the visible range the severity of a skin rash is often obscured when it presents in a patient with pigmented skin. This can result in the patient with pigmented skin being perceived to have less severe disease and being undertreated and prescribed an insufficiently strong medication and can lead to prolonged suffering for the patient.

Given the qualitative and subject nature of visual assessment and the disparities associated with visualizing skin disease in pigmented skin, there is a need for and objective quantitative imaging technology that can assess skin disease across the full spectrum of pigmentation.

Bruises are similarly typically assessed by visual inspection and are subject to similar issues. That is, bruise assessment is typically a qualitative, visual assessment and bruises are less noticeable to an assessor on darker, more pigmented skin. This can lead to under-assessing of the extent and severity of bruising, particularly in non-white patients.

This background section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

### BRIEF SUMMARY

One aspect of this disclosure is a pigment insensitive imaging system for dermatological inspection of a portion of a body of a subject. The pigment insensitive imaging system includes an imaging device operable to capture images of the portion of the body of the subject in a range of wavelengths of light that includes at least some non-visible wavelengths, an illumination and filtering subsystem positioned to illuminate the portion of the body of the subject and provide reflected light in a plurality of different passbands within the range of wavelengths from the portion of the body of the subject to the imaging device, and a controller communicatively coupled to the imaging device and the illumination and filtering subsystem. Each passband of the plurality of different passbands being centered on a different, non-visible wavelength within the range of wavelengths. The controller includes a memory and a processor. The memory stores instructions executable by the processor that program the controller to operate the imaging device and the illumination and filtering subsystem to capture a plurality of images of the portion of the body of the subject, each image of the plurality of images being an image at a different passband of the plurality of different passbands within the range of wavelengths, combine the plurality of images to create a pseudocolor image, and output the pseudocolor image.

Another aspect of this disclosure is a method of capturing images of a portion of a body of a subject for pigment insensitive dermatological inspection using an imaging device operable to capture images in a range of wavelengths of light that includes at least some non-visible wavelengths. The method includes operating the imaging device to capture a plurality of images of the portion of the body of the subject, each image of the plurality of images being an image at a different passband of a plurality of different passbands within the range of wavelengths, each passband of the plurality of different passbands being centered on a different, non-visible wavelength within the range of wavelengths. The plurality of images are combined to create a pseudocolor image and the pseudocolor image is output.

According to another aspect of this disclosure, a method of pigment insensitive dermatological inspection of a portion of a body of a subject using an imaging device operable to capture images in a range of wavelengths of light that includes at short-wave infrared (SWIR) wavelengths includes operating the imaging device to capture a first image, a second image, and a third images of the portion of the body of the subject. The first image is obtained at a first passband centered on a first SWIR wavelength within the range of wavelengths, the second image is obtained at a second passband centered on a second SWIR wavelength within the range of wavelengths, and the third image is obtained at a third passband centered on a third SWIR wavelength within the range of wavelengths. The first, second and third images are combined to create a combined image and the combined image is output.

Various refinements exist of the features noted in relation to the above-mentioned aspects. Further features may also be incorporated in the above-mentioned aspects. These refinements and additional features may exist individually or in any combination. For instance, various features discussed below in relation to any of the illustrated embodiments may be incorporated into any of the above-described aspects, alone or in any combination.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following figures illustrate various aspects of the disclosure.

FIG. 1 is a simplified block diagram of an example imaging system according to the present disclosure.

FIG. 2 is a simplified block diagram of a computing device that may be used in the imaging systems of the present disclosure.

FIG. 3 is a simplified block diagram of an imaging system that is an embodiment of the example imaging system shown in FIG. 1.

FIG. 4 is another diagram of the imaging system shown in FIG. 3.

FIG. 5 is a representation of a portable implementation of the imaging system of FIG. 3

FIG. 6 is a photograph of a prototype of the imaging system shown in FIG. 3.

FIG. 7 is a simplified block diagram of an imaging system that is another embodiment of the example imaging system shown in FIG. 1.

FIG. 8 is another diagram of the imaging system shown in FIG. 7.

FIG. 9 is another diagram of the imaging system shown in FIG. 7.

FIG. 10 is a flow diagram of an image processing process that may be used with the imaging systems of the present disclosure.

FIG. 11 is a flow diagram of another image processing process that may be used with the imaging systems of the present disclosure.

FIG. 12 is a graph of the absorbance of various biological material with respect to wavelength of light.

FIG. 13A is a visible light image of a portion of a human body with darker skin under which water has been injected, surrounded by images taken at other wavelengths.

FIG. 13B is a visible light image of a portion of a human body with lighter skin under which water has been injected, surrounded by images taken at other wavelengths.

FIG. 13C is a pseudocolor shortwave infrared (SWIR) image produced from of the portion of the human body with darker skin in FIG. 13A produced by an imaging system of this disclosure.

FIG. 13D is a pseudocolor SWIR image produced from of the portion of the human body with lighter skin in FIG. 13B produced by an imaging system of this disclosure.

FIG. 14 a graph comparing Weber contrast of visible light images and SWIR images as a function of skin pigmentation.

FIG. 15A is a visible light photograph of a portion of a body with lighter skin tone (ITA 53°) and acne.

FIG. 15B is a visible light photograph of a portion of a body with a darker skin tone (ITA −53°) and acne.

FIG. 15C is a pseudocolor SWIR photograph of the same portion of the body and acne as shown in FIG. 15A.

FIG. 15D is a pseudocolor SWIR photograph of the same portion of the body and acne as shown in FIG. 15B.

FIG. 16 is a comparison of several visible light photos and pseudocolor SWIR image of cupping bruises across differently pigmented portions of the skin of mosaic swine.

FIG. 17 is a graph of quantified contrast (ΔE) for visible images and SWIR pseudocolor images of pigmented, nonpigmented, and mixed portions of skin of mosaic swine.

Corresponding reference characters indicate corresponding parts throughout the drawings.

### DETAILED DESCRIPTION

The field of this disclosure relates generally to imaging devices. Specific embodiments of this disclosure relate to devices, systems, and methods for pigmentation-insensitive imaging. Example embodiments in this described with respect primarily to imaging for assessment of skin disease and bruising, but the devices, systems, and methods described herein may be used for any other suitable imaging purpose.

Skin disease leads to inflammation, which is characterized by increased fluid within skin tissue. Embodiments of this disclosure take advantage of this tissue fluid content as a means of facilitating detection of skin inflammation.

Generally, example embodiments acquire images at specific wavelengths that interact more strongly with fluids that are desired to be imaged and that may be associated with the disease, condition, etc. of interest. For example, as noted above cutaneous inflammation is characterized by shifts in tissue fluid. Visible (VIS, 400-700 nm) and near infrared (NIR, 700-1000 nm) imaging exhibits strong melanin and hemoglobin absorption, and weak water absorption, making water effectively invisible in these spectral ranges. Shifts in tissue water content associated with inflammation are therefore not directly visualized in the VIS or NIR. In the short-wave infrared (SWIR, 1000-1700), water emerges as a strongly absorbing chromophore and melanin exhibits decreased absorption. Some example embodiments, image in this range of wavelengths to provide a means for assessing tissue fluid content while being relatively insensitive to melanin, and the tissue fluid content may be correlated to skin inflammation and skin disease.

In some embodiments, the overall approach is to acquire images in rapid succession at separate wavelengths extending from visible range to SWIR and the algorithmically processing the images and recombining the images to create a new pseudocolor image that visualizes inflammation. For example, in one example embodiment, images acquired at 1200 nm, 1300 nm, and 1600 nm are spectrally encoded relative images acquired at 850 nm, and then the 3 separate images are combined as a weighted average into an RGB image, thereby creating a false-color visible image from wavelengths that are invisible to the eye. In example demonstrations, this process enabled the visualization of water injected into the skin in both dark and lightly pigmented subjects that is otherwise imperceptible by eye. Thus, such embodiments provide a tool that will provide a pigmentation-insensitive and objective means for assessing skin inflammation.

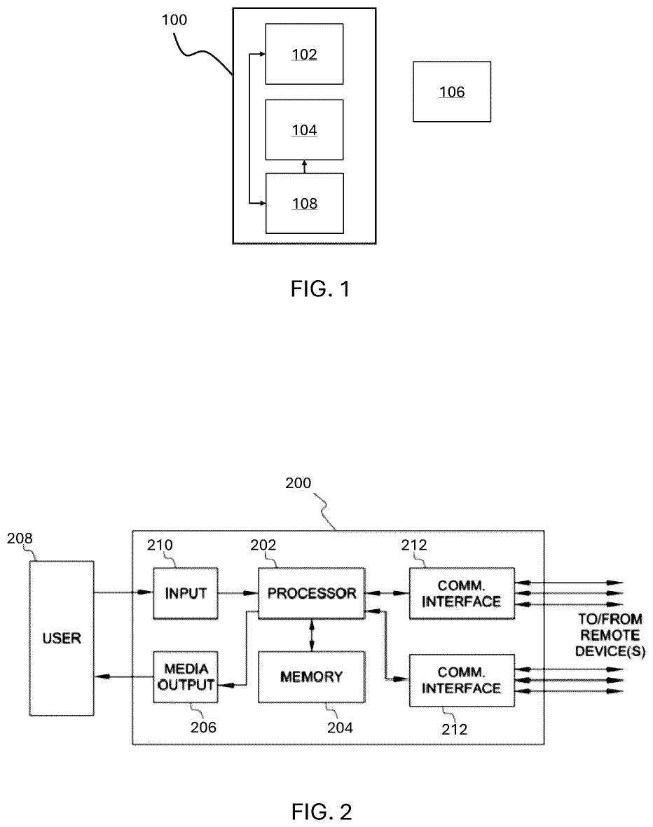

FIG. 1 is a simplified block diagram of an example imaging system 100 for imaging a portion of the body of a subject 106 according to the present disclosure. The imaging system 100 includes an imaging device 102, an illumination and filtering subsystem 104, and a controller 108.

The imaging device 102 is any imaging device suitable for imaging a portion of the body of the subject 106 as described herein. The imaging device may be a camera operable to image across a range of wavelengths of light. The range of wavelengths of light must include at least some non-visible (to humans) wavelengths. Thus, in some embodiments, the imaging device is a camera operable to image in the visible wavelengths from about 400 nanometers (nm) to about 700 nm, in the near infrared (NIR) range of wavelengths from about 700 nm to about 1000 nm, and in the short-wave infrared (SWIR) range of wavelengths from about 1000 nm to about 1700 nm. Other embodiments may only be operable to image in a range of non-visible wavelengths, such as only imaging in the SWIR range, imaging in NIR and SWIR ranges, and the like. The imaging device 102 may include any suitable imaging sensor, controller(s), memory, lens(es), polarizers, or the like (none shown).

The illumination and filtering subsystem 104 is positioned to illuminate the portion of the body of the subject 106 and provide reflected light from the portion of the body of the subject to the imaging device in a plurality of different passbands within the range of wavelengths that the imaging device is operable to image. Each passband of the plurality of different passbands is centered on a different, non-visible wavelength within the range of wavelengths. Examples of the illumination and filtering subsystem will be explained in further detail with reference to other figures below.

The controller 108 is communicatively coupled to the imaging device 102 and the illumination and filtering subsystem 104. The controller broadly includes a memory and a processor, and the memory stores instructions that are executable by the processor. The instructions program the controller to operate the imaging device and the illumination and filtering subsystem to capture a plurality of images of the portion of the body of the subject. In some embodiments, each image of the plurality of images is an image at a different passband of the plurality of different passbands within the range of wavelengths. In an example embodiment, the plurality of images is three images captured in three different passbands. within the range of wavelengths. In an example embodiment, the plurality of images is three images captured in three different passbands. In other embodiments any suitable number of images in any suitable number of passbands may be used. In some preferred embodiments, the plurality of images includes one or more image captured at each passband of three passbands in the range of wavelengths. Thus, in such embodiments, the plurality of images can include any number of images that is a multiple of three. The controller then combines the plurality of images to create a pseudocolor image, and outputs the pseudocolor image.

The controller 108 is programmed, in some embodiments, combine the plurality of images to create the pseudocolor image by assigning the images capture in a first passband to a red image channel, the images capture in a second passband a green image channel, and the images captured in a third passband to a blue image channel.

In some embodiments, the non-visible wavelengths that the imaging device is operable to capture are SWIR wavelengths. In such embodiments, the plurality of passbands includes passbands within the SWIR wavelengths. In some embodiments, the plurality of passbands includes at least three passbands in the SWIR range of wavelengths. Some such embodiments use passbands centered on about 1200 nm, about 1300 nm, and about 1600 nm. Thus, in some embodiments, the pseudocolor image is created by assigning the image from 1200 nm passband to the red image channel, the image from the 1300 nm passband to the green image channel, and the image from the 1600 nm passband to the blue image channel. Other embodiments may additionally or alternatively use passbands in other ranges of wavelengths. In particular, the passbands used may be selected depending on the light absorbance of an item of interest at various wavelengths.

In some embodiments, the controller 108 is programmed to normalize the captured images relative to a normalization image captured in a normalization passband centered on a wavelength shorter than the center wavelength of the other passbands. For example, when the plurality of passbands comprises passbands centered on SWIR wavelengths, the normalization image may be an image captured in a passband centered on a visible light wavelength, an NIR wavelength, or the like.

Turning to FIG. 2, an example configuration of a computing device 200 that may be used as or as part of the controller 108 and/or any other computers, computing device, controllers, or the like described herein is shown. The computing device 200 includes a processor 202, a memory 204, a media output component 206, an input device 210, and communications interfaces 212. Other embodiments include different components, additional components, and/or do not include all components shown in FIG. 2.

The processor 202 is configured for executing instructions. In some embodiments, executable instructions are stored in the memory 204. The processor 202 may include one or more processing units (e.g., in a multi-core configuration). As used herein, the term “processor” refers not only to integrated circuits, but also to a controller, a microcontroller, a microcomputer, a programmable loic controller (PLC), an application-specific integrated circuit, a graphic processing unit, and other programmable circuits. The memory 204 may generally be or include memory element(s) including, but not limited to, computer readable medium (e.g., random access memory (RAM)), computer readable non-volatile medium (e.g., a flash memory), a floppy disk, a compact disc-read only memory (CD-ROM), a magneto-optical disk (MOD), a digital versatile disc (DVD) and/or other suitable non-transitory memory elements and is generally any device allowing information such as executable instructions and/or other data to be stored and retrieved. Such memory 204 may generally be configured to store suitable computer-readable instructions that, when implemented by the processor 202, configure, cause, or program the computing device 200 to perform various functions described herein.

The media output component 206 is configured for presenting information to user 208. The media output component 206 is any component capable of conveying information to the user 208. In some embodiments, the media output component 206 includes an output adapter such as a video adapter and/or an audio adapter. The output adapter is operatively connected to the processor 202 and operatively connectable to an output device such as a display device (e.g., a liquid crystal display (LCD), organic light emitting diode (OLED) display, cathode ray tube (CRT), “electronic ink” display, one or more light emitting diodes (LEDs)) or an audio output device (e.g., a speaker or headphones).

The computing device 200 includes, or is connected to, the input device 210 for receiving input from the user 208. The input device is any device that permits the computing device 200 to receive analog and/or digital commands, instructions, or other inputs from the user 208, including visual, audio, touch, button presses, stylus taps, etc. The input device 210 may include, for example, a variable resistor, an input dial, a keyboard/keypad, a pointing device, a mouse, a stylus, a touch sensitive panel (e.g., a touch pad or a touch screen), a gyroscope, an accelerometer, a position detector, or an audio input device. A single component such as a touch screen may function as both an output device of the media output component 206 and the input device 210.

The communication interfaces 212 enable the computing device 200 to communicate with remote devices and systems, such as allowing communication between the controller 108 and the imaging device 102, the illumination and filtering subsystem 104, remote computing devices or servers (not shown), and the like. The communication interfaces 212 may be wired or wireless communications interfaces that permit the computing device to communicate with the remote devices and systems directly or via a network. Wireless communication interfaces 212 may include a radio frequency (RF) transceiver, a Bluetooth® adapter, a Wi-Fi transceiver, a ZigBee® transceiver, a near field communication (NFC) transceiver, an infrared (IR) transceiver, and/or any other device and communication protocol for wireless communication. (Bluetooth is a registered trademark of Bluetooth Special Interest Group of Kirkland, Washington; ZigBee is a registered trademark of the ZigBee Alliance of San Ramon, California.) Wired communication interfaces 212 may use any suitable wired communication protocol for direct communication including, without limitation, USB, RS232, I2C, SPI, analog, and proprietary I/O protocols. In some embodiments, the wired communication interfaces 212 include a wired network adapter allowing the computing device to be coupled to a network, such as the Internet, a local area network (LAN), a wide area network (WAN), a mesh network, and/or any other network to communicate with remote devices and systems via the network. Although two communication devices 212 are shown, the computing device 200 may include more or fewer computing devices.

It should be understood that in some embodiments the computing device 200 does not include or use an input 210 or a media output 206 and a user 208 may not directly interact with the computing device. Rather, the user 208 (or another computing device) may only interact remotely with computing device 200 through the communication interface 212.

Moreover, in some embodiments the computing device 200, or parts thereof, may not be a physical computing device local to the user 208, but instead is cloud based. Thus, for example, the computing device 145 may be a cloud-based computing device or may be a physical computing device 200 using cloud-based storage for all or part of its memory 204, using cloud-based processing instead of local processing for some or all of its processing, or the like. Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. As used herein, the term “cloud computing” and related terms, e.g., “cloud computing devices” refers generally to a computer architecture allowing for the use of multiple heterogeneous computing devices for data storage, retrieval, and processing. The heterogeneous computing devices may use a common network or a plurality of networks so that some computing devices are in networked communication with one another over a common network but not all computing devices. In other words, a plurality of networks may be used to facilitate the communication between and coordination of all computing devices.

In some embodiments, the computing device 200 may be embodied on or may include a desktop computer, a laptop computer, a tablet computer, a mobile phone, a microcontroller, a single board computer, or any other device operable to function as the controller 108 described herein.

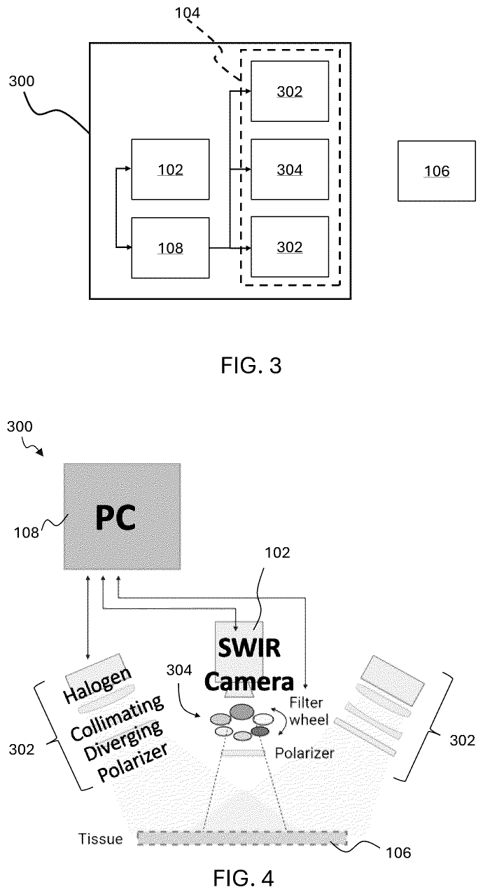

FIG. 3 is a simplified block diagram of an example imaging system 300 based on the imaging system 100 shown in FIG. 1. Unless described otherwise, similar components in the imaging system 300 are similar to and perform similarly to the corresponding components in the imaging system 100. In the imaging system 300, the illumination and filtering subsystem 104 includes lights 302 and a set of passband filters 304.

The lights 302 are operable to illuminate the portion of the body of the subject 106 with light having a spectrum of wavelengths across the range of wavelengths in which the imaging device 102 is operable to capture images. Although two lights are shown, the system 300 may include any number of lights (more or fewer) so long as they are capable of illuminating the subject 106 sufficiently to allow imaging as described herein. Moreover, in some embodiments, the lights illuminate the subject 106 with light in a spectrum that is narrower than the entire spectrum in which the imaging device is operable, but covers the entire range of wavelengths at which images will be captured for performance according to this disclosure. That is, if the images to be captured and combined into a pseudocolor image are all captured in passbands centered on SWIR wavelengths, the lights 302 need only illuminate the portion of the body of the subject 106 with light having a spectrum of wavelengths across the SWIR range of wavelengths (or the portion thereof that includes the plurality of passbands being used). In an example embodiment, the lights 302 are halogen lights.

The set of passband filters 304 includes at least two passband filters, with each passband filter corresponding to a different passband of the plurality of different passbands in which images will be captured. Thus, in an example embodiment using three SWIR passbands, there are at least three passband filters, one centered on 1200 nm, one on 1300 nm, and one on 1600 nm.

The controller 108 is programmed to operate the imaging device 102 and the illumination and filtering subsystem 104 in the system 300 to capture the plurality of images by: operating the lights 302 to illuminate the portion of the body of the subject 106 with light having the spectrum of wavelengths across the range of wavelengths imageable by the imaging device, b) positioning a passband filter of the plurality of passband filters 304 between the imaging device and the subject, c) operating the imaging device to capture an image of the portion of the body of the subject through the positioned passband filter; and d) repeating steps b and c for each passband filter of the plurality of passband filters.

In the example embodiment, the passband filters 304 are a plurality of individual passband filters mounted on a filter wheel controllable by the controller 108, such as a motorized filter wheel. That is, the controller can rotate the filter wheel (or command the filter wheel to rotate) to position a desired passband filter of the plurality of filters on the wheel between the imaging device 102 and the subject 106. Other embodiments use any other suitable system to position one of the passband filters between the imaging device 102 and the subject 106, including robotic arms positioning the filters.

FIG. 4 is a more detailed diagram of an embodiment of the imaging system 300. IN this example, the imaging device 102 is an SWIR camera, the lights 302 are halogen lights with suitable filters, such as collimating, diverging, and polarizing filters, the filters 304 are a filter wheel, and the controller 108 is a personal computer (PC).

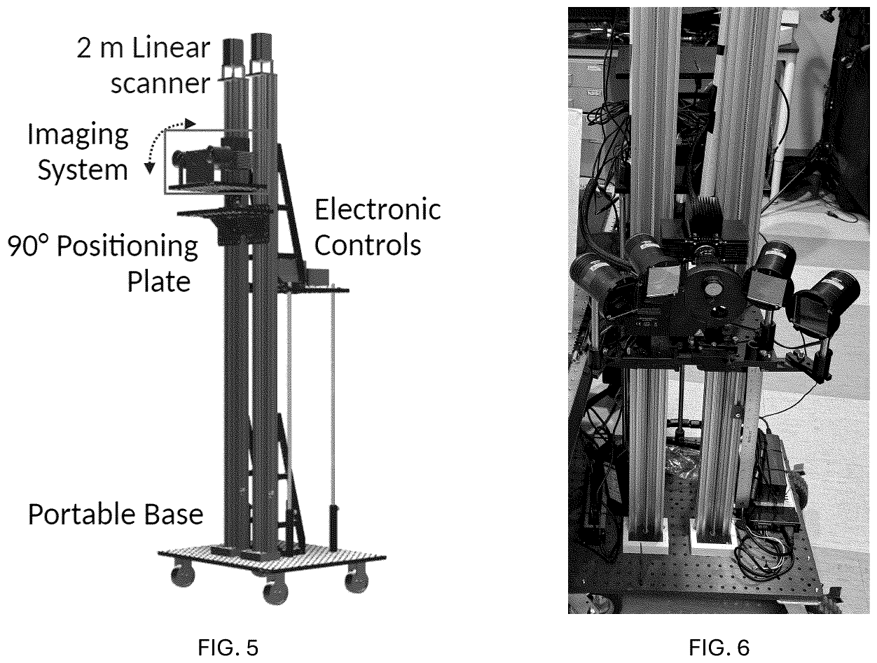

FIG. 5 is a representation of a portable implementation of the imaging system 300 mounted on portable scanner. Such embodiments allow the imaging system to acquiring images and creating pseudocolor images as described above for different portions of the body of the subject (including linear scans of the body of the subject from head to toe). In this example, the portable scanner is a two-meter-tall linear scanner with a portable base, but other embodiments may include any other suitable portable or stationary scanner. FIG. 6 is a photograph of a prototype of such an implementation of the imaging system 300 mounted on a portable scanner.

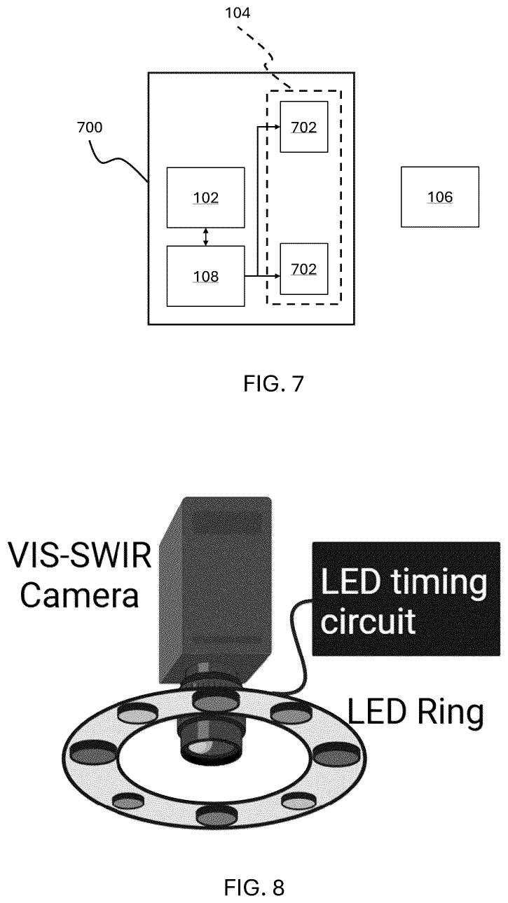

FIG. 7 is a simplified block diagram of an example imaging system 700 based on the imaging system 100 shown in FIG. 1. Unless described otherwise, similar components in the imaging system 300 are similar to and perform similarly to the corresponding components in the imaging system 100. In the imaging system 700, the illumination and filtering subsystem 104 includes a plurality of lights 702. Although only two lights 702 are shown, the system 700 may include any suitable number of lights and generally will include at least one light for each passband at which images are to be collected.

Each of the lights 702 are operable to illuminate the portion of the body of the subject 106. Each light is operable to emit light in one or more wavelengths within a passband range of wavelengths narrower than the range of wavelengths that the imaging device can image. The passband range of each light of the plurality of lights is within a different passband of the plurality of different passbands. Thus, in an embodiment in which images in three SWIR passbands are to be collected, the system 700 would include three lights 702, each one centered on a different SWIR wavelength (e.g., 1200 nm, 1300 nm, and 1600 nm). In some embodiments, two or more lights are used for each light 702, for example to limit shadows, increase the brightness of the illumination, etc. Thus, for example, an embodiment in which images in three SWIR passbands are to be collected may include three lights, one at each of three different SWIR wavelengths (e.g., 1200 nm, 1300 nm, and 1600 nm), six lights, two each at each of three different SWIR wavelengths, nine lights, three at each of three different SWIR wavelengths, or the like.

Because each light 702 illuminates the body of the subject 106 with a spectrum of wavelengths predominately within one passband that is to be imaged, additional filters, such as the passband filters 304 in FIG. 3, are not needed. In some embodiments, the lights are light emitting diodes (LEDs), though any suitable type of lighting operable to emit light in a passband range as described herein may be used. The passband ranges around a selected passband center are generally determined by the particular light chosen for the light 702. Generally, a narrower passband range is preferred, though wider passband ranges may be used in some embodiments. Moreover, the size of the passband ranges may be the same or different for the different lights (i.e., for the different center wavelengths). That is, each light 702 may have a same passband range of, for example, ±3% of its center wavelength, ±35 nm from its center wavelength, or the like. Alternatively, one or more lights 702 may have a passband range that is different from the other lights, such as ±5% of its center wavelength, ±60 nm from its center wavelength, or the like.

In some embodiments cross polarization is used. In such embodiments, one or more polarizer (not shown) is included in front of the lights. Each light may have its own polarizer (aligned with all of the other lights polarizers), or two or more lights may share a polarizer. A camera polarizer (not shown) is also positioned in from of the camera. The camera polarizer is positioned in front of the camera in a cross polarized orientation, which may help reduce surface glare.

The controller 108 is programmed to operate the imaging device 102 and the illumination and filtering subsystem 104 in the system 700 to capture the plurality of images by: a) operating one light of the plurality of lights 702 to illuminate the portion of the body of the subject 103 with light in its passband range of wavelengths, b) operating the imaging device 102 to capture an image of the portion of the body of the subject 106 while illuminated by the one light being operated, and c) repeating steps a and b for each light of the plurality of lights.

By illuminating the subject 106 with individually controllable lights 702, each at different passbands, the system 700 may operate faster than the system 300. This increased speed arises largely from the system 300 needing to physically reposition the filters 304 before taking each image, while the system 700 need only turn off the previously used light 702 and turn on the next light 702 before collecting an image. As a result, the system 700 may capture three or more images in the same time typically taken for one frame of real time video. These three images (one in each of three SWIR passbands) may be combined to form a pseudocolor image and that image may be used as a frame of video in connection with subsequent images. Thus, the system 700 may also be used to acquire, generate, and output real time pseudocolor videos.

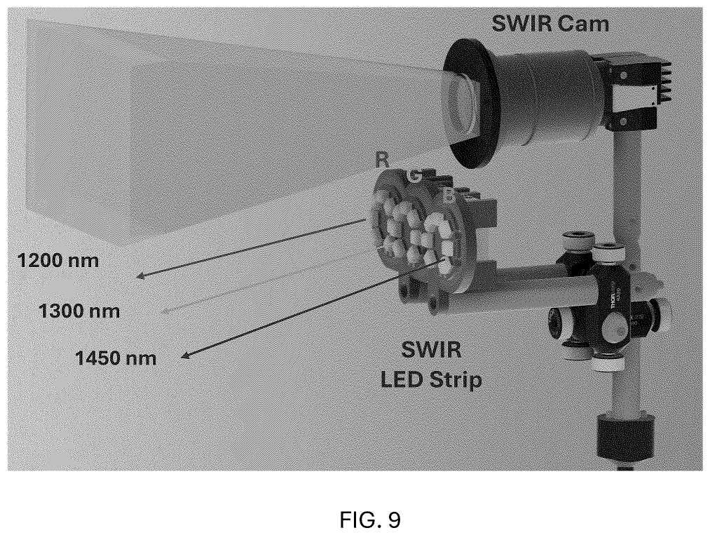

FIGS. 8 and 9 are diagram of two embodiments of the imaging system 700 shown in FIG. 7. In FIG. 8, the imaging device 102 is a VIS-SWIR camera. The lights 702 are arranged in a ring, with the camera imaging the subject through the center of the ring. In this example, there are eight lights in the SWIR wavelengths, two each at each of four different SWIR wavelengths. In this embodiment, the lights are LEDs and an LED timing circuit is used to coordinate lighting and extinguishing the lights 702 in coordination with the imaging by the imaging device 102. In FIG. 9, the imaging device is an SWIR camera, three lights 702 are mounted below the camera to illuminate the subject, and each of the lights 702 includes a plurality of LEDS emitting light at its particular passband range of wavelengths. In this example, the passband ranges of wavelengths are centered on 1200 nm, 1300 nm, and 1450 nm and are assigned to the red, green, and blue imaging channels respectively.

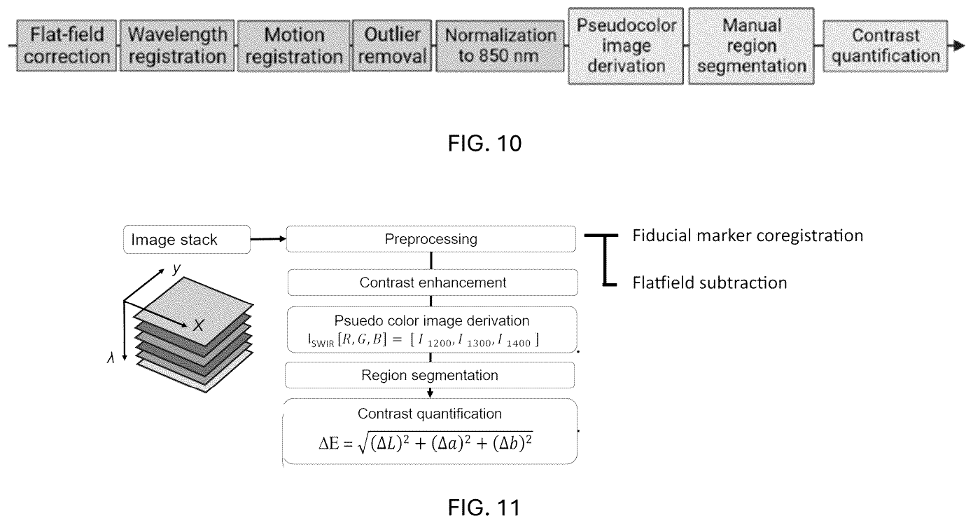

FIG. 10 is a flow diagram of an image processing process that may be used with the imaging systems of the present disclosure. In this image processing algorithm, images undergo preprocessing and spectral encoding and are then utilized to derive improved pseudocolor visualizations and quantifiers of regional contrast. The final two steps are primarily utilized in quantifying results and may not be used in some embodiments. That is, in some embodiments, the pseudocolor images are created and then displayed to a user without any segmentation and/or contrast quantification.

FIG. 11 is a flow diagram of another image processing process that may be used with the imaging systems of the present disclosure. As with the process in FIG. 10, the segmentation and quantification may not be performed in some embodiments.

In some embodiments of any of the above-described systems, the imaging device includes two cameras, a visible light imaging camera and a camera operable to capture light in the SWIR range. A beamsplitter is positioned to split incoming light into two paths, one toward the visible light imaging camera and the other toward the camera operable to capture light in the SWIR range. Thus, a visible light image and SWIR image(s) may be simultaneously captured. This may improve the speed of image acquisition and accuracy of the imaging (because the images are acquired at substantially the same time, there is less time-based difference—e.g., because of movement, changes in lighting, etc.—between images).

The performance and the systems of this disclosure will be illustrated and discussed with respect to the following figures.

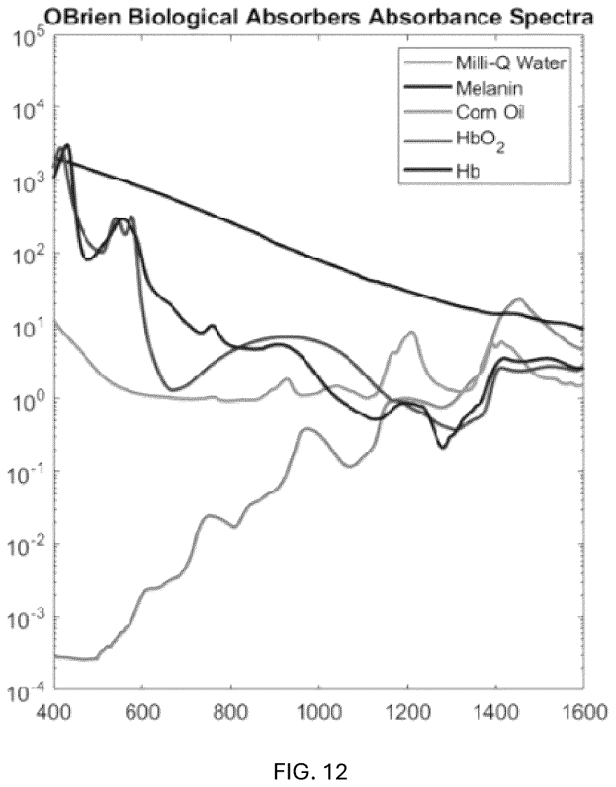

FIG. 12 is a graph of the absorbance of various biological material with respect to wavelength of light. As can be seen water has a very low light absorbance in the visual spectrum and into the NIR spectrum. Thus, in visual and NIR imaging, water is minimally visible. Conversely, melanin has a very high light absorbance in the visible spectrum and into the near NIR spectrum. Thus, in addition to being highly visible, melanin largely masks water in visible/NIR imaging. In the SWIR range of wavelengths, however, waters light absorbance has increased to the point that it is more observable generally and melanin's absorbance has decreased to a point closer to (and in some cases less than) the absorbance of water. Thus, in the SWIR range of wavelengths, water can be more readily imaged, and melanin does not obscure mask water in images of a subject's skin. These characteristics allow the systems of this disclosure to image water movement and bruising while minimizing the impact of skin tone on the inspection, assessment, and documentation of the subject's condition.

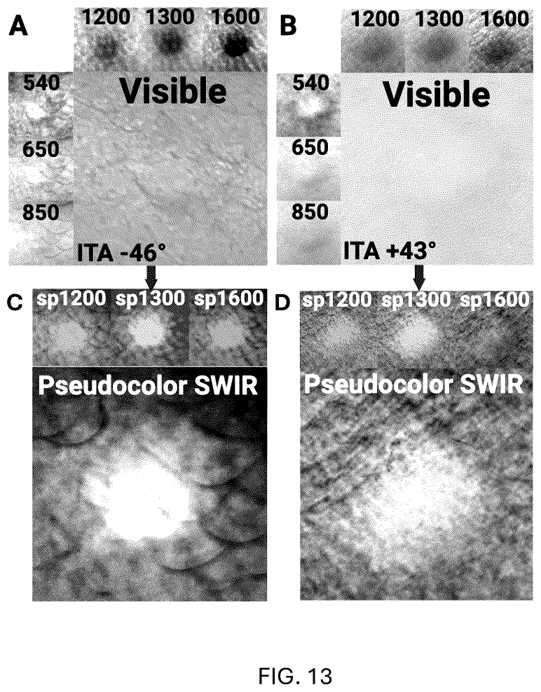

FIGS. 13A-13D include images captured and created using a prototype system according to this disclosure. The prototype system for these figures was based on the system 300 and included bandpass filters centered at 850 nm, 1200 nm, 1300 nm, and 1600 nm. Images captured at 540 nm, 650 nm, and under a visible light spectrum are also shown for comparison.

FIG. 13A is a visible light image of a portion of a human body with darker skin (ITA −46°) under which water has been injected, surrounded by images taken at the other wavelengths. As can be seen, under visible light and up to 850 nm, almost no swelling is visible. In the SWIR images at 1200, 1300, and 1600 nm, the water under the skin is noticeably visible.

FIG. 13B is a visible light image of a portion of a human body with lighter skin (ITA +43°) under which water has been injected, surrounded by images taken at other wavelengths. As can be seen, under visible light and up to 850 nm, swelling is visible to varying degrees. In the SWIR images at 1200, 1300, and 1600 nm, the water under the skin is noticeably visible.

FIG. 13C is a pseudocolor shortwave infrared (SWIR) image produced from of the portion of the human body with darker skin in FIG. 13A produced by an imaging system of this disclosure. FIG. 13D is a pseudocolor SWIR image produced from of the portion of the human body with lighter skin in FIG. 13B produced by an imaging system of this disclosure. The pseudocolor SWIR images were produced by normalizing the respective SWIR wavelength images to their 850 nm image and assigning their 1200 nm image to the red image channel, 1300 nm image to the green image channel, and 1600 nm image to the blue image channel. The water under the skin is clearly visible in the pseudocolor images in both 13C and 13D despite the different in skin tones.

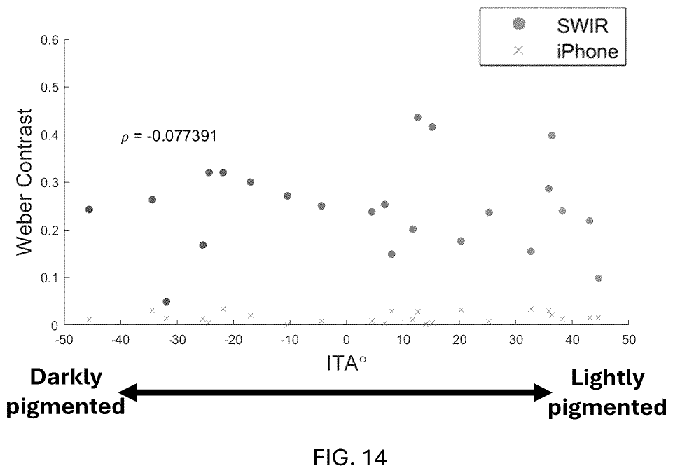

FIG. 14 a graph comparing Weber contrast of visible light images and SWIR images as a function of skin pigmentation. SWIR pseudocolor images and visible light images were produced similar to those in FIG. 13 for a large number of subjects having skin tones ranging from darkly pigmented (˜ITA −50°) to lightly pigmented (˜ITA +50°). The Weber contrast was calculated for each SWIR pseudocolor image and each visible light image. AS can be seen in FIG. 14, the contrast was significantly and consistently higher in the SWIR pseudocolor images as compared to the visible light images.

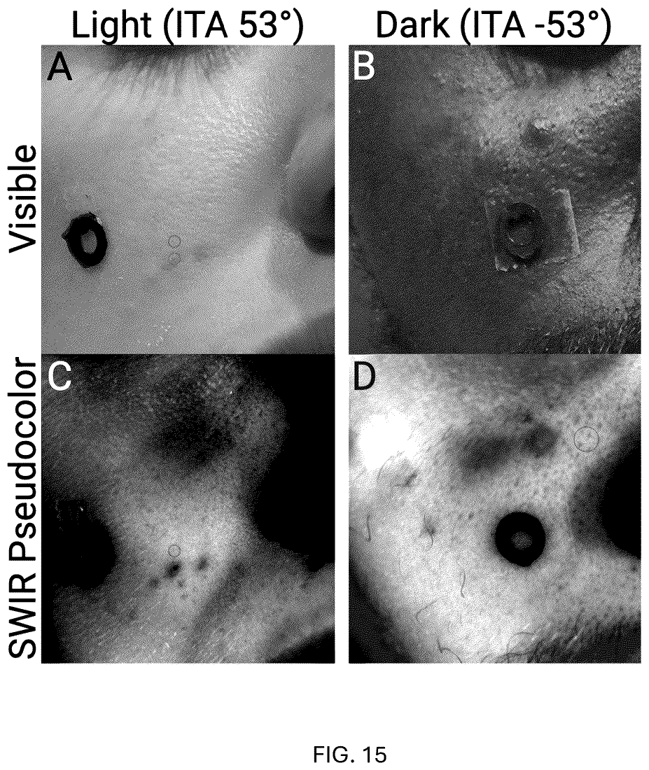

FIGS. 15A-15D include images captured and created using a prototype system according to this disclosure. The prototype system for these figures was based on the system 300 and included bandpass filters centered at 850 nm, 1200 nm, 1300 nm, and 1600 nm. Images captured under a visible light spectrum are also shown for comparison.

FIG. 15A is a visible light photograph of a portion of a body with lighter skin tone (ITA 53°) and acne. FIG. 15B is a visible light photograph of a portion of a body with a darker skin tone (ITA −53°) and acne. FIG. 15C is a pseudocolor SWIR photograph of the same portion of the body and acne as shown in FIG. 15A. FIG. 15D is a pseudocolor SWIR photograph of the same portion of the body and acne as shown in FIG. 15B. For both skin tones, the acne lesions are more clearly visible on the SWIR pseudocolor images (FIGS. 15C and 15D) than in the visible light images (FIGS. 15A and 15B).

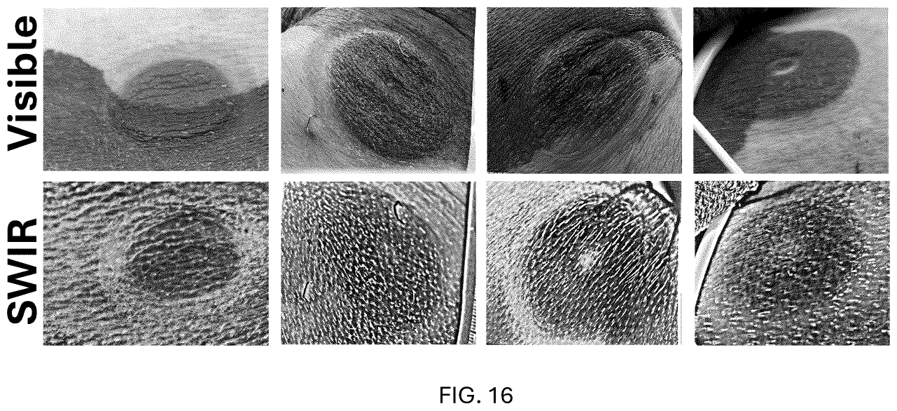

FIG. 16 is a comparison of several visible light photos (upper row) and pseudocolor SWIR images (lower row) of cupping bruises across differently pigmented portions of the skin of mosaic swine. The SWIR images were created using a prototype imaging system based on the imaging system 700. As can be seen in the visible light images, the bruises appear more prominent and easily identifiable on the less/nonpigmented portions of skin and are difficult to see on the darkly pigmented portions of skin. In the corresponding SWIR pseudocolor images, each entire cupping bruise is clearly visible an there is little to no distinction between the visible bruising on the pigmented skin and on the unpigmented skin. This confirms that the example systems of this disclosure may allow accurate identification and classification of bruising regardless of pigmentation of the skin of the subject who has experienced the bruising.

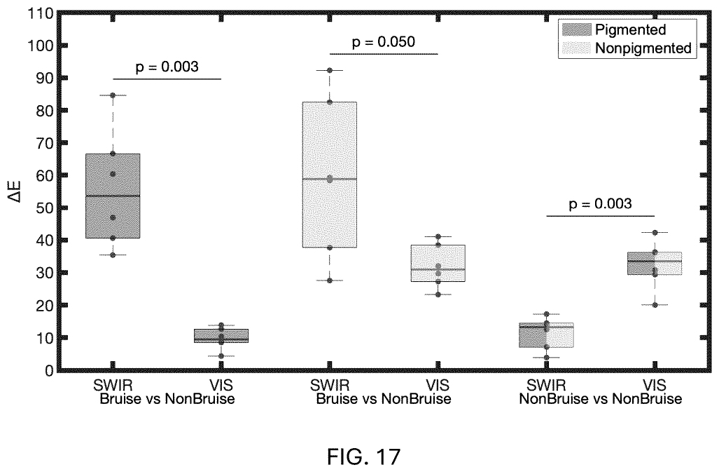

FIG. 17 is a graph of quantified contrast (ΔE) for a set of visible images and SWIR pseudocolor images of pigmented, nonpigmented, and mixed portions of skin of mosaic swine, of which the images in FIG. 16 are a sample. As can be seen, the contrast between bruised skin and non-bruised skin was higher for the SWIR images than for the visible light images for both pigmented and nonpigmented skin. Also notable, for the nonpigmented skin the quantified contrast under visible light is significantly higher than the quantified contrast under visible light for pigmented skin. This difference is greatly reduced in the SWIR images.

Thus, the example imaging systems of this disclosure provide high contrast visualization of cutaneous dermal tissue fluid regardless of skin pigmentation. These systems may overcome the limitations of subjective visual assessment by providing an objective means for equitably assessing the degree of skin inflammation, bruising, and the like in patients spanning the full spectrum of skin pigmentation. The example systems may facilitate the ability to identify and assess disease severity in dermatology patients with, for example, atopic dermatitis, psoriasis, and hidradenitis suppurativa spanning a diverse range of skin pigmentation.

This written description uses examples to disclose the invention, including the best mode, and also to enable any person skilled in the art to practice the invention, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

As used herein, the terms “about,” “substantially,” “essentially” and “approximately” when used in conjunction with ranges of dimensions, concentrations, temperatures or other physical or chemical properties or characteristics is meant to cover variations that may exist in the upper and/or lower limits of the ranges of the properties or characteristics, including, for example, variations resulting from rounding, measurement methodology or other statistical variation.

When introducing elements of the

present disclosure or the embodiment(s) thereof, the articles “a”, “an”, “the” and “said” are intended to mean that there are one or more of the elements. The terms “comprising,” “including,” “containing” and “having” are intended to be inclusive and mean that there may be additional elements other than the listed elements. The use of terms indicating a particular orientation (e.g., “top”, “bottom”, “side”, etc.) is for convenience of description and does not require any particular orientation of the item described.

As various changes could be made in the above constructions and methods without departing from the scope of the disclosure, it is intended that all matter contained in the above description and shown in the accompanying drawing[s] shall be interpreted as illustrative and not in a limiting sense.

What is claimed is:

1. A pigment insensitive imaging system for dermatological inspection of a portion of a body of a subject, the imaging system comprising:
an imaging device operable to capture images of the portion of the body of the subject in a range of wavelengths of light that includes at least some non-visible wavelengths;
an illumination and filtering subsystem positioned to illuminate the portion of the body of the subject and provide reflected light in a plurality of different passbands within the range of wavelengths from the portion of the body of the subject to the imaging device, each passband of the plurality of different passbands being centered on a different, non-visible wavelength within the range of wavelengths; and
a controller communicatively coupled to the imaging device and the illumination and filtering subsystem, the controller including a memory and a processor, the memory storing instructions executable by the processor that program the controller to:
operate the imaging device and the illumination and filtering subsystem to capture a plurality of images of the portion of the body of the subject, each image of the plurality of images being an image at a different passband of the plurality of different passbands within the range of wavelengths;
combine the plurality of images to create a pseudocolor image; and
output the pseudocolor image.

2. The pigment insensitive imaging system of claim 1, wherein:
the illumination and filtering subsystem comprises a light operable to illuminate the portion of the body of the subject with light having a spectrum of wavelengths across the range of wavelengths and including all of the plurality of different passbands, and a plurality of passband filters, each passband filter corresponding to a different passband of the plurality of different passbands; and
the controller is programmed to operate the imaging device and the illumination and filtering subsystem to capture the plurality of images by:
a) operating the light to illuminate the portion of the body of the subject with light having the spectrum of wavelengths across the range of wavelengths;
b) positioning a passband filter of the plurality of passband filters between the imaging device and the subject;
c) operating the imaging device to capture an image of the portion of the body of the subject through the positioned passband filter; and
d) repeating steps b and c for each passband filter of the plurality of passband filters.

3. The pigment insensitive imaging system of claim 2, wherein the illumination and filtering subsystem comprises a motorized filter wheel controllable by the controller and each passband filter of the plurality of passband filters is mounted at a different location on the motorized filter wheel.

4. The pigment insensitive imaging system of claim 1, wherein:
the illumination and filtering subsystem comprises a plurality of lights, each light of the plurality of lights being operable to emit light in one or more wavelengths within a passband range of wavelengths narrower than the range of wavelengths, the passband range of each light of the plurality of lights being within a different passband of the plurality of different passbands; and
the controller is programmed to operate the imaging device and the illumination and filtering subsystem to capture the plurality of images by:
a) operating one light of the plurality of lights to illuminate the portion of the body of the subject with light in its passband range of wavelengths;
b) operating the imaging device to capture an image of the portion of the body of the subject while illuminated by the one light being operated; and
c) repeating steps a and b for each light of the plurality of lights.

5. The imaging system of claim 4, wherein the plurality of lights comprise light emitting diodes (LEDs).

6. The imaging system of claim 1, wherein the non-visible wavelengths comprise short-wave infrared (SWIR) wavelengths.

7. The imaging system of claim 6, wherein the plurality of different passbands comprise three passbands and the controller is programmed to combine the plurality of images to create a pseudocolor image by assigning the images capture in a first passband of the three passbands to a red image channel, the images capture in a second passband of the three passbands to a green image channel, and the images capture in a third passband of the three passbands to a blue image channel.

8. The pigment insensitive imaging system of claim 7, wherein the first passband is centered on about 1200 nanometers (nm), the second passband is centered on about 1300 nm, and the third passband is centered on about 1600 nm.

9. The pigment insensitive imaging system of claim 7, wherein the controller is further programmed to capture a normalization image in a normalization passband that is centered at a wavelength shorter than the SWIR wavelengths.

10. The pigment insensitive imaging system of claim 9, wherein the controller is further programmed to normalize the images captured in the first passband, the second passband, and the third passband to the normalization image.

11. A method of capturing images of a portion of a body of a subject for pigment insensitive dermatological inspection using an imaging device operable to capture images in a range of wavelengths of light that includes at least some non-visible wavelengths, the method comprising:
operating the imaging device to capture a plurality of images of the portion of the body of the subject, each image of the plurality of images being an image at a different passband of a plurality of different passbands within the range of wavelengths, each passband of the plurality of different passbands being centered on a different, non-visible wavelength within the range of wavelengths;
combining the plurality of images to create a pseudocolor image; and
outputting the pseudocolor image.

12. The method of claim 11, wherein operating the imaging device to capture the plurality of images comprises:
a) illuminating the portion of the body of the subject with light having a spectrum of wavelengths across the range of wavelengths and including all of the plurality of different passbands;
b) positioning a passband filter of a plurality of passband filters between the imaging device and the subject, each passband filter of the plurality of passband filters corresponding to a different passband of the plurality of different passbands;
c) operating the imaging device to capture an image of the portion of the body of the subject through the positioned passband filter; and
d) repeating steps b and c for each passband filter of the plurality of passband filters.

13. The method of claim 11, wherein operating the imaging device to capture the plurality of images comprises:
a) illuminating the portion of the body of the subject with light in a passband range of wavelengths within the range wavelengths and narrower than the range of wavelengths;
b) operating the imaging device to capture an image of the portion of the body of the subject while illuminated by light in the passband range of wavelengths; and
c) repeating steps a and b one or more times for different passband ranges of wavelengths than in previous performances of steps a and b.

14. The method of claim 11, wherein the non-visible wavelengths comprise short-wave infrared (SWIR) wavelengths, the plurality of different passbands comprise three passbands and combining the plurality of images to create the pseudocolor image comprises assigning the images capture in a first passband of the three passbands to a red image channel, the images capture in a second passband of the three passbands to a green image channel, and the images capture in a third passband of the three passbands to a blue image channel.

15. The method of claim 14, further comprising:
capturing a normalization image in a normalization passband that is centered at a wavelength shorter than the SWIR wavelengths; and
normalizing the images captured in the first passband, the second passband, and the third passband to the normalization image before combining the plurality of images to create the pseudocolor image.

16. A method of pigment insensitive dermatological inspection of a portion of a body of a subject using an imaging device operable to capture images in a range of wavelengths of light that includes at short-wave infrared (SWIR) wavelengths, the method comprising:
operating the imaging device to capture a first image, a second image, and a third images of the portion of the body of the subject, the first image being obtained at a first passband centered on a first SWIR wavelength within the range of wavelengths, the second image being obtained at a second passband centered on a second SWIR wavelength within the range of wavelengths, and the third image being obtained at a third passband centered on a third SWIR wavelength within the range of wavelengths;
combining the first, second and third images to create a combined image; and
outputting the combined image.

17. The method of claim 16, wherein the combined image comprises a pseudocolor image, and combining the first, second and third images to create the combined image comprises assigning the first image to a red image channel, the second image to a green image channel, and the third image a blue image channel.

18. The method of claim 17, further comprising:
illuminating the portion of the body of the subject with light fully within the first passband when capturing the first image;
illuminating the portion of the body of the subject with light fully within the second passband when capturing the second image; and
illuminating the portion of the body of the subject with light fully within the third passband when capturing the third image.

19. The method of claim 17, further comprising:
illuminating the portion of the body of the subject with light having a spectrum of wavelengths across the range of wavelengths and including light within the first, second, and third passbands;
filtering the light reflected from the portion of the body of the subject before it enters the imaging device with a first passband filter centered on the first SWIR wavelength when capturing the first image;
filtering the light reflected from the portion of the body of the subject before it enters the imaging device with a second passband filter centered on the second SWIR wavelength when capturing the second image; and
filtering the light reflected from the portion of the body of the subject before it enters the imaging device with a third passband filter centered on the third SWIR wavelength when capturing the third image.

20. The method of claim 17, further comprising capturing a normalization image in a normalization passband that is centered at a wavelength shorter than SWIR wavelengths; and
normalizing the first, second, and third images to the normalization image before combining the first, second and third images to create the combined image.
